(19) United States
(12) Patent Application Publication
(10) Pub. No.: US 2002/0044915 A1
(43) Pub. Date: Apr. 18, 2002

(54) Whitening cosmetics containing extracts from Ecklonia cava

(76) Inventors: Bong-Ho Lee, Taejon (KR); Byung-Wook Choi, Taejon (KR); Geon-Seek Ryu, Taejon (KR); Hyeon-Cheol Shin, Taejon (KR); Key-Jung Kang, Taejon (KR); Jeong-Ha Kim, Seoul (KR); Hae-Young Jeong, Chungchurngbook-do (KR)

Correspondence Address: ABELMAN FRAYNE & SCHWAB, 150 East 42nd Street, New York, NY, 10017, US

(21) Appl. No.: 09/895,516

(22) Filed: Jun. 28, 2001

(30) Foreign Application Priority Data
Aug. 23, 2000 (KR) 2000-48933

(51) Int. Cl. A61K 7/135; A61K 35/78

(52) U.S. Cl. 424/63; 424/725

(57) ABSTRACT

Disclosed are whitening cosmetics containing extracts from Ecklonia cava. More specifically, Ecklonia cava which has been dried and crushed into powder, is immersed in a solvent, deposited to extract whitening materials, then, the materials are distilled under reduced pressure to obtain extracts from Ecklonia cava, which are added to whitening cosmetics. The whitening cosmetics containing the extracts from Ecklonia cava of the present invention exhibit excellent whitening effect because of strongly inhibiting tyrosinase activity.

### BACKGROUND OF THE INVENTION

1. Field of the Invention

The present invention relates to whitening cosmetics containing extracts from Ecklonia cava. More specifically, the present invention relates to whitening cosmetics containing whitening materials extracted from Ecklonia cava, which exhibits excellent whitening effect because of inhibiting tyrosinase activity.

2. Description of the Prior Art

In general, there are various reasons for the darkening of skin color, the main reason being exposure to ultraviolet rays. When skin is exposed to ultraviolet rays, melanin is synthesized in melanocytes, which is a type of skin cell, and released to darken skin color. In the process of melanin synthesis in melanocytes, tyrosinase reacts on tyrosine, which is a substrate for tyrosinase, in the cell to produce Dopaquinone and then it goes through spontaneous reaction and sequential enzyme reaction of Dopaquinone to provide a copolymeric black pigment, melanin (Jose Neptuno Rodriguez-Lopez, Jose Tudela, Ramon varon, Francisco Garcia-Carmons, and Francisco Garcia-canovas, J. Biol. Chem., Vol. 267, No. 34, 3810 (1992); Pilar Aroca, Kazunori Urabe, Takeshi Kobayashi, Katsuhiko Tsukamoto, and Vincent J. Hearing, J. Biol. Chem., Vol. 268, No. 34, 25650 (1993)).

In order to prevent skin color from being darkened, it is most simple and general to inhibit a part of the melanin generating steps, and thus to reduce the production of melanin. Conventionally, an intensive research for developing an agent with tyrosinase-inhibiting activity has been focused on ascorbic acid, kojic acid, arbutin, hydroquinone, and extracts from various plants including extract from morus bark etc (U.S. Pat. No. 5,063,056).

Among these, kojic acid forms a chelate with a copper ion at the active site of tyrosinase to inhibit the enzyme activity. Although showing good performance, said material causes stability problems in the process of blending it into cosmetic products. Further, hydroquinone is undesirable for the use in cosmetic materials because it strongly irritates the skin. Furthermore, the use of hydroquinone in cosmetic products is now prohibited in Korea and other countries. As for Ascorbic acid, the excellent effectiveness thereof as a whitening cosmetic cannot be obtained due to low stability of the molecule itself and low whitening effect. Materials extracted from various plants show whitening effects, but mostly such materials have substantial inhibitory effects on tyrosinase activity when used in high concentrations. When used in relatively low concentrations, tyrosinase inhibition activity is hardly detectable. Accordingly, an economic benefit is not brought about.

There is thus an urgently recognized need for a novel whitening material having a strong whitening effect and high stability, capable of alleviating the problems of conventional whitening materials.

### SUMMARY OF THE INVENTION

Leading to the present invention, the intensive and thorough research on materials extracted from Ecklonia cava, carried out by the present inventors aiming to avoid the problems encountered in the prior arts, resulted in the finding that the materials extracted from Ecklonia cava have an excellent inhibitory effect in tyrosinase activity so that the materials when being used in whitening cosmetics show high stability and superior whitening effect.

Accordingly, it is an object of the present invention to provide whitening cosmetics containing materials extracted from Ecklonia cava, which are of strong whitening effect and high stability.

### DESCRIPTION OF THE INVENTION

Whitening materials extracted from Ecklonia cava used in the present invention show excellent effects in inhibiting tyrosinase activity so that the materials can be mainly used in whitening cosmetics.

Ecklonia cava used in the present invention is perennial seaweeds distributed in the sea near Cheju Island and Japan. Ecklonia cava reaches its full growth in spring. The stem of Ecklonia cava is cylindrical in shape, and grows 1 m tall or higher. Its center portion is somewhat large, exhibiting multilocular textures in early years, but becoming hollow in later years. A single layer of slime vessel is circularly arranged beneath the cortex of the stem. Upper portions of the stem become gradually flat, with pinnate leaves growing on both sides of the stem, on which leaflets of alternate pinnate leaves grow. The center leaves are 3 to 5 mm thick. The surface of the leaves has no rumples. Although the color of Ecklonia cava is brown, it turns black on drying. Ecklonia cava has leather-like texture and grows in deeper sea zones. Its young bodies are prevalent in spring, and the body is about 5 to 10 cm long in stem, about 5 mm long in diameter of stem, about 20 to 30 cm long in center leaves, and 4 cm long in width. Every two years, its spores are released from Sordaria fimicola formed in center leaves from autumn to winter, and then the center leaves are lost, thus only stem remains. Soon, old center leaves which had been present at the top of the stem are replaced by new center leaves. Accordingly, 2 or 3 years are required to grow sufficiently mature Ecklonia cava.

In the present invention, the extracts from Ecklonia cava are obtained as follows:

Whole parts of Ecklonia cava obtained from the sea near Cheju Island are washed with pure water, dried out of direct sunlight and then crushed into powder. This powdered Ecklonia cava is immersed in a solvent to obtain extracts containing whitening ingredients therefrom. Preferable examples of the solvent are water, methanol, ethanol, butanol, ethyl acetate, acetonitrile, acetone, 1,3-butylene glycol, aqueous solutions thereof, and mixtures thereof. The solvent is used at an amount of 1 to 15 weights, preferably 5 to 10 weights based on the dry weight of Ecklonia cava. Optionally, said extraction procedure may be carried out at least twice in order to increase the yield, wherein a different organic solvent may be used in each repetition step. It is preferred that a depositing process is used during the extraction procedure. In addition to the depositing process, other known processes including stirring etc. may be used. The depositing process is conducted at 10 to 50° C. for one hour to 15 days, preferably at 15 to 35° C. for 12 to 24 hours. The solution extracted through the above procedure is concentrated under reduced pressure using a distiller equipped with a cooling condenser to obtain materials extracted from Ecklonia cava to be used in the present invention.

For eliminating undesired materials and the solvent from the extracted materials, use can be made of separation and concentration instruments such as a centrifuge and a rotary evaporation concentrator. The extracts obtained through said procedure can be directly used in various fields of applications, so that they are economically favorable because of requiring no additional processes.

Materials extracted from Ecklonia cava have been used in various applications, but they have not been used in whitening cosmetics so far. The whitening cosmetics containing the materials extracted from Ecklonia cava show a superior whitening effect and high stability.

Examples of the cosmetics containing materials extracted from Ecklonia cava of the present invention are cosmetics for skin care (skin softener, cream, essence, cleansing foam, cleansing water, facial pack, body oil etc.), cosmetics for make-up (foundation, lipstick, mascara, make-up base etc.), and cosmetics for hair (shampoo, rinse, hair conditioner, hair gel etc.). The whitening cosmetics can be prepared by a conventional method for preparing cosmetic products, including components of the following Formulations.

The whitening cosmetics of the present invention contain 0.001 to 5% by weight, preferably 0.005 to 1% by weight of extracts from Ecklonia cava based on the dry weight thereof. If the amount of the extracts is less than 0.001% by weight, the whitening effect is reduced. On the other hand, if the amount thereof exceeds 5% by weight, an economic disadvantage is brought about.

A better understanding of the present invention may be obtained in light of the following examples, experimental examples, comparative examples and formulations which are set forth to illustrate, but are not to be construed to limit the present invention.

### EXAMPLE 1

1 kg of Ecklonia cava sample, which had been washed with refined water, dried and crushed to a grain size of 2 to 3 cm, was immersed in 9 L of water, extracted at 15 to 25° C. for 12 to 24 hours and then filtered through filter paper of Wattman No. 5. The extracts were concentrated under reduced pressure using a distiller (Buich, Switzerland) equipped with a cooling condenser to obtain 387 g (dry weight) of the dried extracts from Ecklonia cava.

### EXAMPLES 2-15

These example were carried out in the same manner as in Example 1 to obtain the dried extracts from Ecklonia cava, except that solvents used in the extraction procedure as shown in the following Table 1 were used. The dry weights are given in Table 1, below.

TABLE 1

| Example Nos. | Solvents | Dry Weight (g) |
|---|---|---|
| 1 | Water | 387 |
| 2 | 10% Ethanol | 362 |
| 3 | 20% Ethanol | 325 |
| 4 | 30% Ethanol | 350 |
| 5 | 40% Ethanol | 337 |
| 6 | 50% Ethanol | 350 |
| 7 | 60% Ethanol | 312 |
| 8 | 70% Ethanol | 300 |
| 9 | 80% Ethanol | 238 |
| 10 | 90% Ethanol | 263 |
| 11 | 100% Ethanol | 51 |
| 12 | 100% Methanol | 178 |
| 13 | Acetone | 14 |
| 14 | Ethyl Acetate | 10 |
| 15 | Butanol | 15 |

### EXPERIMENTAL EXAMPLE 1

### Tyrosinase Activity Inhibitory Effect of Extracts from Ecklonia cava According to the Present Invention

Tyrosinase (from Sigma Co., USA) extracted from fungus and refined was used in the Example. Tyrosine, which is a substrate of tyrosinase was dissolved in 0.05 M sodium phosphate buffer solution (pH 6.8) and used at a concentration of 0.1 mg/ml.

Each of the dried extracts from Ecklonia cava obtained in Examples 1-15 was dissolved in aqueous 1,3-butylene glycol solution at high concentration, and diluted in sodium phosphate buffer solution to concentrations of 30, 60, 150, and 300 μ/ml and used as test samples. 0.5 ml of the tyrosine solution was introduced into a test tube, and 0.5 ml of the samples was added thereto, and then incubated at 37° C. for 10 minutes. Thereafter, the tube was added with 0.5 ml of 250 U/ml tyrosinase solution and again incubated at 37° C. for 10 minutes. As a control group, 0.5 ml of buffer solution was added instead of each extract and the same reaction was performed. After the reaction was complete, the reaction was quenched by placing the test tube on ice to inhibit further enzyme action in the system. Absorbance was measured at a wavelength of 475 nm by using a spectrophotometer.

Inhibitory effect on tyrosinase activity of the extracts was determined by the following equation. The results are given in Table 2, below.

TABLE 2
$\% inhibition of tyrosinase activity = 100 - \frac{Absorbance of extract}{Absorbance of control} \times 100$

| | Inhibition Ratio (%) | | | |
|---|---|---|---|---|
| Run | 10 (μg/ml) | 20 (μg/ml) | 50 (μg/ml) | 100 (μg/ml) |
| 1 | 12.34 | 14.56 | 15.82 | 39.56 |
| 2 | 11.39 | 16.46 | 26.27 | 35.13 |
| 3 | 16.60 | 31.58 | 47.37 | 61.94 |
| 4 | 27.12 | 35.88 | 56.21 | 72.32 |
| 5 | 30.06 | 45.57 | 61.39 | 74.37 |
| 6 | 35.44 | 50.32 | 70.25 | 82.00 |
| 7 | 18.04 | 36.71 | 58.86 | 72.47 |
| 8 | 39.23 | 48.34 | 67.96 | 82.04 |
| 9 | 40.88 | 50.28 | 66.30 | 78.45 |
| 10 | 43.65 | 56.637 | 73.76 | 82.87 |
| 11 | 38.67 | 58.56 | 77.07 | 88.12 |
| 12 | 29.79 | 47.61 | 64.63 | 81.38 |
| 13 | 53.72 | 63.30 | 78.99 | 87.50 |
| 14 | 31.91 | 48.94 | 54.52 | 66.49 |
| 15 | 23.94 | 47.61 | 58.24 | 72.87 |

As shown in the above table 2, the materials extracted from Ecklonia cava exhibit a high inhibitory effect on tyrosinase activity.

### COMPARATIVE EXAMPLE 1

### Comparison of Tyrosinase Inhibitory Effect: Extracts from Ecklonia cava vs Conventional Whitening Materials

The tyrosinase activity inhibitory effect of the conventional whitening materials was measured through the same methods as shown in the Experimental Example 1 to obtain IC₅₀ values, which is the concentration of the sample required to inhibit 50% of tyrosinase activity. For comparison with the extracts from Ecklonia cava of the present invention, extracts from morus bark (comparative example 1), extracts from liquorice (comparative example 2), and extracts from schizandra (comparative example 3) were used, wherein the extracts were obtained in the same manner as described in the Example 1. Also, as conventional chemical materials were used albutin (comparative example 4), kojic acid (comparative example 5) and ascorbic acid (comparative example 6). The results are given in Table 3, below.

TABLE 3

| Used Whitening Materials | IC₅₀ (μg/ml) |
|---|---|
| Extracts from Ecklonia cava (Example 10) | 14.89 |
| Extracts from Mours bark (C. Example 1) | 17.87 |
| Extracts from Liquorice (C. Example 2) | 52.28 |
| Extracts from Schizandra (C. Example 3) | 29.04 |
| Arbutin (C. Example 4) | 68.32 |
| Kojic Acid (C. Example 5) | 6.21 |
| Ascorbic acid (C. Example 6) | 70.65 |

From the results of the above table 3, although the extracts from Ecklonia cava used in the present invention are crude extracts, it is found that the extracts of the present invention have superior tyrosinase activity inhibitory effects to those of conventional whitening materials.

### EXPERIMENTAL EXMAPLE 2

### Inhibitory Effect on Melanin Synthesis of Extracts from Ecklonia cava

As melanocytes, commercially available B-16 melanoma (ATCC CRL 6323) cell line derived from mouse was used.

The melanoma cell line was inoculated into DMEM culture medium containing 4.5 g/l glucose, 10% serum and 1% antibiotic agent, and cultivated at 37° C. under a condition of 5% CO₂ for 24 hours. After cultivation was completed, the cultivation solution was added with 1 ml of phosphate buffer solution (PBS) containing 0.02% EDTA and 0.05% trypsin to isolate cells, which was then inoculated into the same medium as described above in a 50 ml T-flask and cultivated for 48 hours. Thereafter, when the number of cells reached 4.88×10⁶, extracts from Ecklonia cava diluted in DMEM medium at a concentration of 50, 100, 150, and 200 μg/ml were added to the cultivated cells, and the mixture was cultivated at 5% CO₂ and 37° C. for 3 days. After cultivation, culture medium was thoroughly removed, and the cells were isolated through the same method as above, which were then centrifuged for 5 minutes to collect pure cells. The obtained cells were treated with 5% trichloroacetate (TCA), stirred, and centrifuged. Then, precipitated melanin was washed with phosphate buffer solution, and treated with 1N NaOH to dissolve melanin therein. Absorbance at 475 nm was measured. Melanin concentration was determined from a standard concentration curve of synthetic melanin (from SIGMA Co., USA). The results are shown in the following Table 4.

TABLE 4

| Concentration of Extracts | Inhibition Ratio |
|---|---|
| From Ecklonia cava (μg/ml) | On Melanin Synthesis (%) |
| 50 | 74.8 |
| 100 | 87.9 |
| 150 | 92.6 |
| 200 | 94.3 |

The results show that the extracts from Ecklonia cava of the present invention are very effective in inhibiting melanin synthesis.

### FORMULATION 1

An exemplary formula of a skin softener containing extracts from Ecklonia cava of the present invention is shown in Table 5, below.

TABLE 5

| Components | Content (weight %) |
|---|---|
| Extracts from Ecklonia cava | 0.1 |
| 1,3-butylene glycol | 6.0 |
| Sodium hyaluronate | 2.0 |
| Glycerin | 4.0 |
| PEG 4000 | 1.0 |
| Polysorbate 20 | 0.5 |
| Ethanol | 10.0 |
| Preservatives | Proper amount |
| Benzophenone-9 | 0.05 |
| Fragrance | Proper amount |
| Refined water | Remainder |
| Total | 100 |

An exemplary formula of a milk lotion containing extracts from Ecklonia cava of the present invention is shown in Table 6, below.

TABLE 6

| Components | Content (weight %) |
|---|---|
| Extracts from Ecklonia cava | 0.1 |
| Stearic acid | 0.4 |
| 1,3-Butylene Glycol | 6.0 |
| Cetostearyl alcohol | 1,2 |
| Glycerin | 4.0 |
| Glyceryl Stearate | 1.0 |
| Triethanolamine | 0.25 |
| Tocopheryl Acetate | 3.0 |
| Liquid Paraffin | 5.0 |
| Squalane | 3.0 |
| Macadamia Nut Oil | 2.0 |
| Polysorbate 60 | 1.5 |
| Sorbitan sesquioleate | 0.6 |
| Carboxy vinyl polymer | 0.15 |
| Preservatives | Proper amount |
| Fragrance | Proper amount |
| Refined water | Remainder |
| Total | 100 |

### FORMULATION 3

An exemplary formula of a nutrient cream containing extracts from Ecklonia cava of the present invention is shown in Table 7, below.

TABLE 7

| Components | Content (weight %) |
|---|---|
| Extracts from Ecklonia cava | 0.1 |
| Vaselin | 7.0 |
| Cetostearyl alcohol | 2.5 |
| Glyceryl stearate | 2.0 |
| Stearic acid | 1.5 |
| Liquid paraffin | 10.0 |
| Wax | 2.0 |
| Polysorbate 60 | 1.5 |
| Sorbitan sesquioleate | 0.8 |
| Squalane | 3.0 |
| 1,3-Butylene glycol | 6.0 |
| Glycerin | 4.0 |
| Triethanolamine | 0.5 |
| Tocopheryl acetate | 0.1 |
| Preservatives | Proper amount |
| Fragrance | Proper amount |
| Refined water | Remainder |
| Total | 100 |

### FORMULATION 4

An exemplary formula of an essence containing extracts from Ecklonia cava of the present invention is shown in Table 8, below.

TABLE 8

| Components | Content (weight %) |
|---|---|
| Extracts from Ecklonia cava | 0.1 |
| Glycerin | 10.0 |
| PEG 1500 | 2.0 |
| Allantoin | 0.1 |
| Panthenol | 0.3 |
| EDTA | 0.02 |
| Bezophenone-9 | 0.04 |
| Hydroxy ethyl cellurose | 0.1 |
| Sodium hyaluronate | 8.0 |
| Carboxy vinyl polymer | 0.2 |
| Triethanolamine | 0.18 |
| Octyldodeces-25 | 0.6 |
| Ethanol | 6.0 |
| Preservatives, Fragrance, Pigment | Proper amount |
| Refined water | Remainder |
| Total | 100 |

### FORMULATION 5

An exemplary formula of a massage cream containing extracts from Ecklonia cava of the present invention is shown in Table 9, below.

TABLE 9

| Components | Content (weight %) |
|---|---|
| Extracts from Ecklonia cava | 0.1 |
| Glyceryl stearate | 2.0 |
| Cetostearyl alcohol | 2.5 |
| Stearic acid | 1.0 |
| Polysorbate 60 | 1.5 |
| Sorbitan stearate | 0.6 |
| Isostearyl isostearate | 5.0 |
| Squalene | 5.0 |
| Mineral oil | 35.0 |
| Dimethicone | 1.0 |
| Xanthan gum | 0.1 |
| Hydroxyethyl cellurose | 0.12 |
| Glycerin | 6.0 |
| Triethanol amine | 0.5 |
| Preservatives, Fragrance, Pigment | Proper amount |
| Refined water | Remainder |
| Total | 100 |

### FORMULATION 6

An exemplary formula of a facial pack containing extracts from Ecklonia cava of the present invention is shown in Table 10, below.

TABLE 10

| Components | Content (weight %) |
|---|---|
| Extracts from Ecklonia cava | 0.1 |
| Polyvinyl alcohol | 15.0 |
| Cellurose gum | 0.15 |
| Glycerin | 3.0 |
| PEG 1500 | 2.0 |
| Panthenol | 0.4 |
| Allantoin | 0.1 |
| Ethanol | 6.0 |
| PEG 40 hydrogenated castor oil | 0.3 |
| Preservatives, Fragrance, Pigment | Proper amount |
| Refined water | Remainder |
| Total | 100 |

### EXPERIMENTAL EXAMPLE 3

### Evaluation of Whitening Effect of the Cosmetic Products According to the Present Invention

Whitening effect of the cosmetic products according to the present invention was evaluated by clinical testing.

A nutrient cream prepared according to Formulation 3 containing 0.1% by weight of extracts from Ecklonia cava, and a nutrient cream prepared by the same Formulation but in which extracts from Ecklonia cava have been replaced with water, were used for the test. The test group consisting of 40 female subjects, aged 20 to 35, was randomly divided into two groups, and one group was applied with the proper amount of the cream containing extracts from Ecklonia cava on their upper arms twice everyday, in the morning and at the evening, while the other group was applied with the cream not containing extracts from Ecklonia cava. After finishing the practical use test for one month, whitening effect was evaluated with a calorimeter by examining a change of skin color before and after the test. The results are shown in Table 11, below.

TABLE 11
WHITENING EFFECT OF COSMETIC PRODUCTS CONTAINING
EXTRACTS FROM Ecklonia cava

| Test materials | ΔL |
|---|---|
| Cosmetic containing extracts from Ecklonia cava | 3.6 |
| Cosmetic not containing extracts from Ecklonia cava | 0.8 |

ΔL = skin color after the test - skin color before the rest

From the above results, it can be seen that the extracts from Ecklonia cava show a clear whitening effect.

Accordingly, whitening cosmetics containing extracts from Ecklonia cava of the present invention showed excellent whitening effect because of inhibiting tyrosinase activity.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A whitening cosmetic containing an extract from Ecklonia cava.

2. The whitening cosmetic as defined in claim 1, wherein the extract from Ecklonia cava is obtained by immersing dry Ecklonia cava powder in 1 to 15 weights of an extraction solvent based on the dry weight of Ecklonia cava, depositing or stirring the solution at 10 to 50° C. for 1 hour to 15 days to extract a whitening material, and then distilling the whitening material under reduced pressure.

3. The whitening cosmetic as defined in claim 2, wherein the extraction solvent is selected from the group consisting of water, methanol, ethanol, butanol, ethyl acetate, acetonitrile, acetone, 1,3-butylene glycol, aqueous solutions thereof, and mixtures thereof.

4. The whitening cosmetic as defined in claim 1, wherein the extract from Ecklonia cava is contained at an amount of 0.001 to 5% by weight based on the dry weight of the cosmetic.

5. The whitening cosmetic as defined in claim 4, wherein the extract from Ecklonia cava is contained at an amount of 0.001 to 1% by weight based on the dry weight of the cosmetic.
